(19) United States
(12) Patent Application Publication
(10) Pub. No.: US 2024/0050227 A1
(43) Pub. Date: Feb. 15, 2024

(54) PROSTHETIC HEART VALVE WITH FRAMES

(71) Applicant: CARDIOVALVE LTD., Or Yehuda (IL)

(72) Inventor: Gil Hacohen, Ramat Gan (IL)

(73) Assignee: CARDIOVALVE LTD., Or Yehuda (IL)

(21) Appl. No.: 18/384,139

(22) Filed: Oct. 26, 2023

Related U.S. Application Data
(63) Continuation of application No. 17/734,472, filed on May 2, 2022, now Pat. No. 11,839,541.
Application No. 17/734,472 is a continuation of application No. 17/479,418, filed on Sep. 20, 2021, now Pat. No. 11,351,026.
Application No. 17/479,418 is a continuation of application No. 16/811,732, filed on Mar. 6, 2020, now Pat. No. 11,141,268.
Application No. 16/811,732 is a continuation of application No. 16/532,945, filed on Aug. 6, 2019, now Pat. No. 10,660,751.
Application No. 16/532,945 is a continuation of application No. 16/388,038, filed on Apr. 18, 2019, now Pat. No. 10,548,726.
Application No. 16/388,038 is a continuation of application No. 16/183,140, filed on Nov. 7, 2018, now Pat. No. 10,610,359.
Application No. 16/183,140 is a division of application No. 15/188,507, filed on Jun. 21, 2016, now Pat. No. 10,231,831.
Application No. 15/188,507 is a continuation of application No. 14/522,987, filed on Oct. 24, 2014, now abandoned.
Application No. 14/522,987 is a continuation of application No. 12/961,721, filed on Dec. 7, 2010, now Pat. No. 8,870,950.
(60) Provisional application No. 61/283,819, filed on Dec. 8, 2009.

(51) Int. Cl. A61F 2/24

(52) U.S. Cl. CPC A61F 2/2418; A61F 2/2409; A61F 2/2412; A61F 2/2445; A61F 2/2454; A61F 2/2439; A61F 2220/0016; A61F 2/2457

(57) ABSTRACT

Apparatus and methods are provided for use with a prosthetic valve that is designated for implantation at a patient's native heart valve, including a valve ring having a plurality of ring segments, each of the segments being hingedly coupled to an adjacent segment at a pivot joint. The valve ring is placed adjacent to a surface of the native heart valve, the prosthetic valve having been coupled to the valve ring. In an expanded state thereof, the valve ring defines a ring, all of the pivot joints being disposed in a plane that is perpendicular to a longitudinal axis of the ring. The valve ring is foldable into a shape that has a generally circular cross-section that defines and surrounds at least in part a central lumen, by folding the segments with respect to each other, at the pivot joints. Other embodiments are also described.

### CROSS REFERENCES TO RELATED APPLICATIONS

The present application is a Continuation of U.S. Ser. No. 17/734,472 to HaCohen, filed May 2, 2022, and entitled “PROSTHETIC HEART VALVE WITH UPPER SKIRT,” which published as US 2022/0257376 and which is a Continuation of U.S. Ser. No. 17/479,418 to HaCohen, filed Sep. 20, 2021, and entitled “ROTATION-BASED ANCHORING OF AN IMPLANT,” (now U.S. Pat. No. 11,351,026) and which is a Continuation of U.S. Ser. No. 16/811,732 to HaCohen, filed Mar. 6, 2020, and entitled “PROSTHETIC HEART VALVE WITH UPPER AND LOWER SKIRTS,” (now U.S. Pat. No. 11,141,268) which published as US 2020/0205969 and which is a Continuation of U.S. Ser. No. 16/532,945 to HaCohen, filed Aug. 6, 2019, and entitled “PROSTHETIC HEART VALVE WITH UPPER SKIRT” (now U.S. Pat. No. 10,660,751), which is a Continuation of U.S. Ser. No. 16/388,038 to HaCohen, filed Apr. 18, 2019, and entitled “ROTATION-BASED ANCHORING OF AN IMPLANT” (now U.S. Pat. No. 10,548,726), which is a Continuation of U.S. Ser. No. 16/183,140 to HaCohen, filed Nov. 7, 2018, and entitled “FOLDING RING PROSTHETIC HEART VALVE” (now U.S. Pat. No. 10,610,359), which is a Divisional of U.S. Ser. No. 15/188,507 to HaCohen, filed Jun. 21, 2016, and entitled “FOLDING RING IMPLANT FOR HEART VALVE” (now U.S. Pat. No. 10,231,831), which is a Continuation of U.S. Ser. No. 14/522,987 to HaCohen, filed Oct. 24, 2014, and entitled “IMPLANT FOR ROTATION-BASED ANCHORING,” which published as US 2015/0045880 (abandoned) and which is a Continuation of U.S. Ser. No. 12/961,721 to HaCohen, filed Dec. 7, 2010, and entitled “ROTATION-BASED ANCHORING OF AN IMPLANT” (now U.S. Pat. No. 8,870,950), which claims the benefit of U.S. Provisional Patent Application 61/283,819, entitled “FOLDABLE HINGED PROSTHETIC HEART VALVE,” to Hacohen, filed Dec. 8, 2009, which is incorporated herein by reference.

### FIELD OF EMBODIMENTS OF THE INVENTION

Embodiments of the present invention relates in general to valve replacement. More specifically, embodiments of the present invention relate to replacement of an atrioventricular valve and prosthetic valve therefor.

### BACKGROUND

Ischemic heart disease causes regurgitation of a heart valve by the combination of ischemic dysfunction of the papillary muscles, and the dilatation of the ventricle that is present in ischemic heart disease, with the subsequent displacement of the papillary muscles and the dilatation of the valve annulus.

Dilation of the annulus of the valve prevents the valve leaflets from fully coapting when the valve is closed. Regurgitation of blood from the ventricle into the atrium results in increased total stroke volume and decreased cardiac output, and ultimate weakening of the ventricle secondary to a volume overload and a pressure overload of the atrium.

### SUMMARY OF EMBODIMENTS

In some applications of the present invention, a prosthetic heart valve structure is provided that collapses and expands by means of one or more valve pivot joints. The prosthetic valve structure is typically designated for implantation in a native atrioventricular valve site of a heart of a patient, although for some applications, the prosthetic valve structure is designated for implantation at the aortic or tricuspid valve. The prosthetic valve structure comprises an annular ring portion that is designated for placement adjacent to the ventricular surface of the native valve of the patient. This annular ring portion comprises the valve pivot joints, which facilitate collapsing of the prosthetic valve structure for transcatheter advancement of the valve toward the heart of the patient. Additionally, the annular portion of the prosthetic valve structure is coupled to a plurality of anchors which are configured to grasp the native chordae tendineae of the heart of the patient. These anchors comprise generally curved prong structures which, in an expanded state of the prosthetic valve structure, are aligned circumferentially along the annular ring portion of the prosthetic valve structure (generally perpendicular to a radius of the annular ring portion). Once the annular ring portion is positioned adjacent to the ventricular surface of the native mitral valve, the prosthetic valve structure is rotated, in order for the anchors to engage the native chordae tendineae. During the engaging, portions of the native chordae tendineae are gathered between each anchor and a respective portion of the annular ring. This engaging provides support to the prosthetic valve structure as it is positioned in and replaces the native valve. Additionally, the prosthetic valve structure comprises ventricular and atrial skirts which provide flush positioning of the prosthetic valve in the native valve.

There is therefore provided, in accordance with some applications of the present invention, apparatus for use with a prosthetic valve that is designated for implantation at a native heart valve of a patient, including:
a valve ring having a plurality of ring segments, each of the segments being hingedly coupled to at least one adjacent segment at a pivot joint,
the valve ring being configured:
   to be placed adjacent to a surface of the native heart valve, the prosthetic valve having been coupled to the valve ring,
   in an expanded state thereof, to define a ring, all of the pivot joints being disposed in a plane that is perpendicular to a longitudinal axis of the ring, and
   to be foldable from the expanded state into a shape that has a generally circular cross-section that defines and surrounds at least in part a central lumen, by folding the segments with respect to each other, at the pivot joints.

For some applications, the segments of the ring are configured to become at least partially twisted due to the ring being folded from the expanded state.

For some applications, the prosthetic valve includes a trileaflet valve, and the ring has a number of ring segments that is a multiple of six.

For some applications, the ring has exactly six segments.

For some applications, the prosthetic valve includes a bileaflet valve, and the ring has a number of ring segments that is a multiple of four.

For some applications, the ring has exactly four segments.

There is further provided, in accordance with some applications of the present invention, a method for use with a prosthetic valve that is designated for implantation at a native heart valve of a patient, including:
placing in a vicinity a surface of the native heart valve, a valve ring that is coupled to the valve, while the valve ring is in a folded state thereof,
   the ring having a plurality of ring segments, each of the segments being hingedly coupled to at least one adjacent segment at a pivot joint,
   in the folded state thereof, the ring having a shape that has a generally circular cross-section that defines and surrounds at least in part a central lumen;
expanding the ring such that all of the pivot joints become disposed in a plane that is perpendicular to a longitudinal axis of the ring, by applying a force to at least some of the pivot joints; and
when the ring is in an expanded state thereof, positioning the ring adjacent to a surface of the native valve.

For some applications, applying the force to some of the pivot joints includes pushing on pivot joints that are disposed on a proximal side of the ring, while the ring is in the folded state thereof.

For some applications, the native valve includes a native mitral valve, and positioning the ring adjacent to the surface of the valve includes positioning the ring adjacent to a ventricular surface of the native mitral valve.

There is additionally provided, in accordance with some applications of the present invention, apparatus for use with a prosthetic valve that is designated for implantation at a native mitral valve of a patient, including:
an annular ring configured to be placed at a ventricular surface of the native mitral valve, the prosthetic valve having been coupled to the annular ring; and
at least one anchor disposed circumferentially with respect to the annular ring so as to define a space between the anchor and the annular ring.

For some applications, the anchor is configured to grasp a portion of native chordae tendineae of a heart of the patient by the annular ring being rotated.

For some applications, the annular ring is configured to be collapsible.

For some applications, the ring includes a plurality of ring segments, each of the segments being hingedly coupled to at least one adjacent segment at a pivot joint, and the ring is configured to be collapsed by folding the segments with respect to each other, at the pivot joints.

There is further provided, in accordance with some applications of the present invention, a method, including:
positioning an annular ring portion of a prosthetic valve structure at a ventricular surface of a native heart valve of a patient; and
grasping a portion of native chordae tendineae of a heart of the patient by rotating the annular ring portion of the prosthetic valve structure.

In some applications of the present invention, grasping the portion of the native chordae tendineae includes facilitating placing the portion of the native chordae tendineae in a space between a segment of the annular ring portion and an anchor disposed circumferentially with respect to the segment of the annular ring portion.

In some applications of the present invention, positioning the annular ring portion includes:
transcatheterally advancing the prosthetic valve structure toward the native valve of the patient in a collapsed state thereof; and
expanding the prosthetic valve structure from the collapsed state.

In some applications of the present invention, expanding the prosthetic valve structure includes pivoting a plurality of segments of the annular ring portion at respective pivot joints that couple together adjacent segments of the annular ring portion.

The present invention will be more fully understood from the following detailed description of embodiments thereof, taken together with the drawings, in which:

### BRIEF DESCRIPTION OF THE DRAWINGS

FIG. 1 is a schematic illustration of an exploded view of a prosthetic heart valve, in accordance with some applications of the present invention;

FIGS. 2A-B are schematic illustrations of the prosthetic heart valve of FIG. 1 in an assembled, collapsed state, in accordance with some applications of the present invention;

FIG. 3 is a schematic illustration of the prosthetic heart valve of FIG. 1 in an assembled, expanded state, in accordance with some applications of the present invention; and

FIG. 4 is a schematic illustration of respective views of the prosthetic heart valve of FIG. 1, while anchors of the valve are anchoring the valve to chordae tendineae of a subject, in accordance with some applications of the present invention.

FIG. 5 is a schematic illustration of an exploded view of a prosthetic heart valve, in accordance with some applications of the present invention;

FIG. 6 is a schematic illustration of the prosthetic heart valve of FIG. 5 in an assembled, expanded state, in accordance with some applications of the present invention; and

FIG. 7 is a schematic illustration of the prosthetic heart valve of FIG. 5 in an assembled, collapsed state, in accordance with some applications of the present invention.

### DETAILED DESCRIPTION OF THE EMBODIMENTS

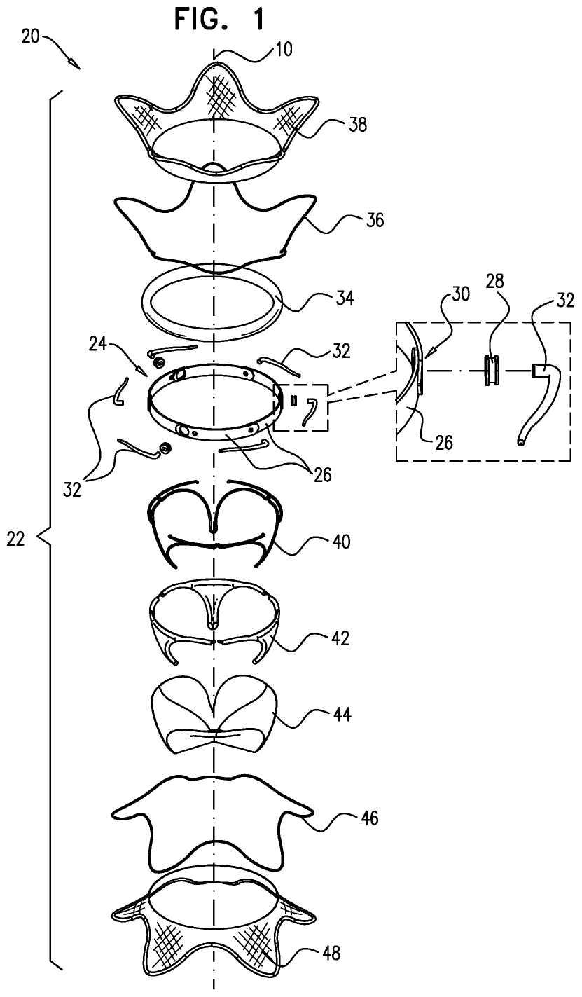

Reference is now made to FIGS. 1-4, which are schematic illustrations of an expandable and collapsible prosthetic valve structure 20, in accordance with some applications of the present invention. The prosthetic valve structure is configured for implantation in and replacement of a native atrioventricular valve of a patient. Typically, the prosthetic valve structure is configured for implantation in and replacement of a native mitral valve of the patient.

The prosthetic valve structure comprises an annular valve ring 24, which comprises a plurality of curved metal segments 26 and a plurality of pivot joints 30 which facilitate the collapsing and expanding of the prosthetic valve structure. The annular valve ring is typically surrounded by a valve ring fabric sleeve 34 comprising a braided mesh of fabric, e.g., Dacron. This sleeve promotes fibrosis following implantation of the prosthetic valve structure in the native valve of the patient. The annular valve ring is coupled to a prosthetic valve that includes a plurality of valve leaflets 44. The valve leaflets are coupled to a flexible valve leaflet frame 40 (e.g., comprising nitinol, by way of illustration and not limitation), which is, in turn, coupled to a valve leaflet frame fabric 42. Typically, the valve leaflet frame fabric (e.g., a fabric comprising Dacron) is coupled to (for example, sutured to) valve ring fabric sleeve 34.

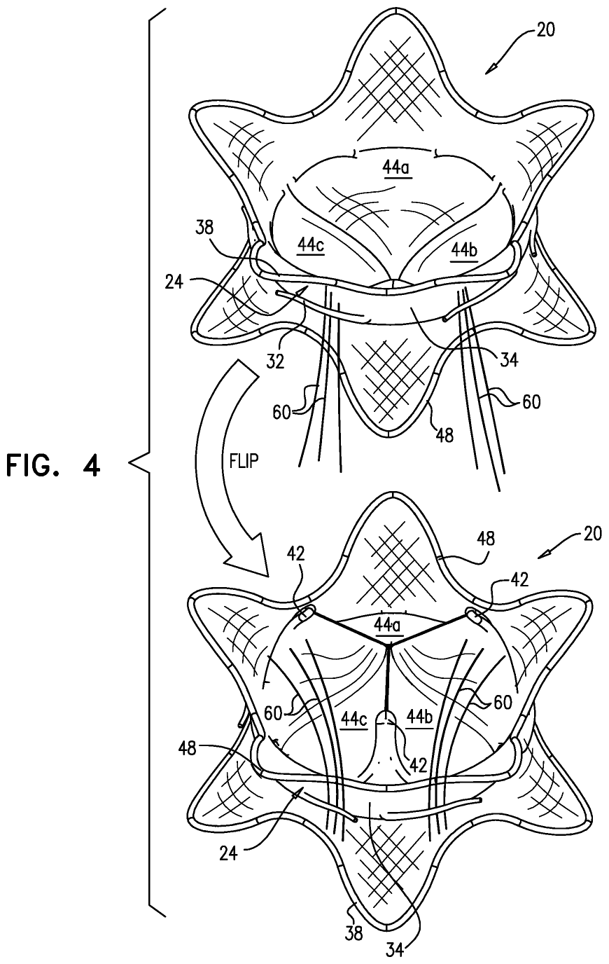

For embodiments in which the prosthetic valve is designated to replace the native mitral valve of the patient, the prosthetic valve comprises three artificial or tissue-based leaflets 44a, 44b, and 44c (shown in FIG. 4), which are coupled to the inner perimeter of the annular valve ring. For example, the leaflets may include pericardium, a polymer, and/or other materials, as would be obvious to one skilled in the art.

The annular valve ring portion of the prosthetic valve structure is coupled: (1) at a first surface thereof to an upper skirt, which comprises an upper skirt fabric 38 coupled to a flexible upper skirt frame 36, and (2) at a second surface thereof to a lower skirt, which comprises a lower skirt fabric 48 coupled to a flexible lower skirt frame 46. Typically, the upper and lower frames comprise a flexible material, e.g., nitinol by way of illustration and not limitation. Typically, when the prosthetic valve structure is implanted in the expanded state, as shown in FIG. 4:
(a) the annular valve ring portion is configured to be disposed at a ventricular surface of the native valve,
(b) the upper skirt is designated to rest against an atrial portion of the native mitral valve, and
(c) the lower skirt is designated to rest against a ventricular surface of the native valve and to push radially the native leaflets of the native valve.

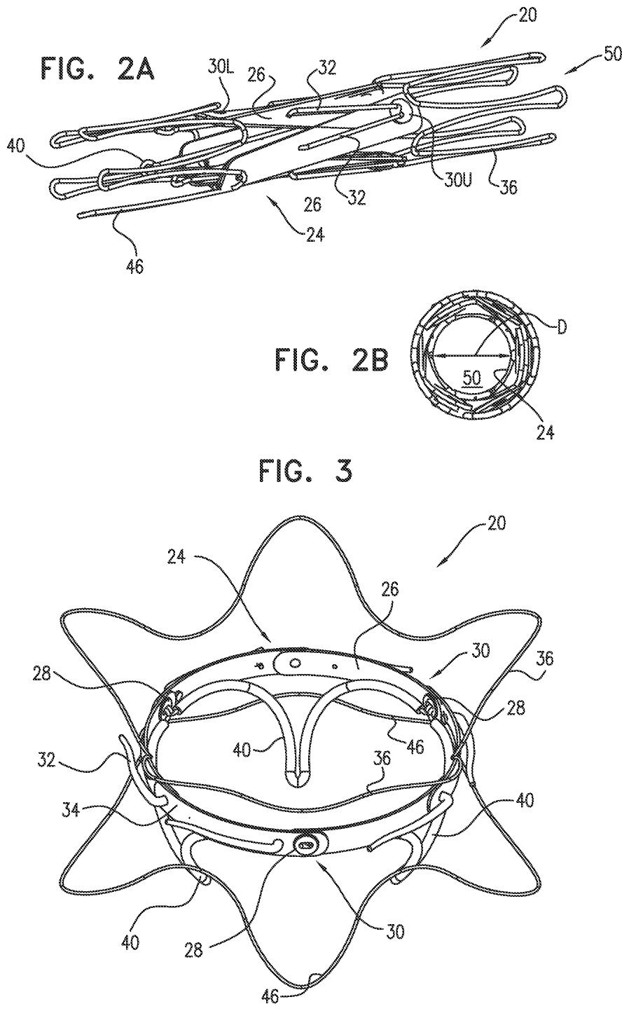

FIG. 1 shows components 22 of valve structure 20 in an exploded view. Each segment 26 of annular valve ring 24 is coupled at its respective ends to respective ends of adjacent segments via a hinge. For example, as shown, the hinge may include a connecting element 28 that is inserted into holes in the ends of the adjacent segments, such that the adjacent segments form a pivot joint 30. The pivot joints of the ring portion enable the entire prosthetic valve structure to pivot into a collapsed state, the collapsed state being shown in FIGS. 2A-B. Typically, the valve comprises: (a) three “upper” valve pivot joints 30U (shown in FIG. 2A) which are disposed at 120 degrees along the annular valve ring and are near the upper skirt frame 36, in the collapsed state of the valve, shown in FIG. 2A; and (b) three “lower” valve pivot joints 30L (shown in FIG. 2A) also disposed with a separation therebetween of 120 degrees, alternating with the upper valve pivot joints. The lower valve pivot joints are near the lower skirt frame 46, in the collapsed state of the valve as shown in FIG. 2A. The upper valve pivot joints are exposed at a proximal portion of the valve in the collapsed state of the valve (i.e., adjacent to an upper skirt region of the prosthetic valve structure, as shown in FIG. 2A), such that a physician is able to push on the upper valve pivot joints with a pushing tool, as described hereinbelow. Typically, when the ring is in its expanded state, all of the pivot joints are disposed in a plane that is perpendicular to longitudinal axis 10 of ring 24.

The pivot joints enable the prosthetic valve structure to collapse to form a shape having a generally circular cross-section that defines and surrounds at least in part a central lumen 50, as shown in FIGS. 2A-B. Typically, the pivot joints enable the valve to assume an outer diameter of less than 10 mm, e.g., less than 6 mm (by way of illustration and not limitation), in its collapsed state, as shown in FIG. 2A. Further typically, central lumen 50 (which is defined by ring 24 in its collapsed state) has a cross-sectional length (e.g., diameter D, shown in FIG. 2B) of between 3 mm and 5 mm, the length being measured in a plane that is perpendicular to longitudinal axis 10 of the valve (shown in FIG. 1).

Typically, when used with a trileaflet valve, ring 24 includes six segments 26, such that there are a total of six pivot joints 30 (of which three are upper pivot joints 30U, and three are lower pivot joints 30L), and such that each of the leaflets is disposed between two adjacent upper pivot joints 30U, or two adjacent lower pivot joints 30L. For some applications, the ring includes twelve (or another multiple of six) pivot joints, such that each of the leaflets of a trileaflet valve is disposed between two non-adjacent upper pivot joints 30U, or two non-adjacent lower pivot joints 30L. For some applications, ring 24 is used with a bileaflet valve. For such applications, the ring may include four, eight, or twelve segments 26, such that there are a corresponding number of pivot joints, and such that each of the leaflets is disposed between two of the upper pivot joints or two of the lower pivot joints.

Each of segments 26 of ring 24 is configured to become twisted when the ring is folded, as shown in FIG. 2A. For some applications, due to shape-memory properties of the segments, the segments facilitate the expansion of the ring, since the segments are pre-shaped in non-twisted shapes.

In the collapsed state of the valve, the valve leaflet frame, the valve leaflets, the upper skirt, and the lower skirt are also collapsed. Typically, the valve is configured such that the expansion of the ring causes each of the aforementioned portions to become expanded automatically.

In order to deploy prosthetic valve structure 20 inside the heart, the physician pushes the upper pivot joints 30U distally, using a pushing tool. The pushing of the upper pivot joints enables annular valve ring 24 to expand radially in order for the prosthetic valve structure to assume an expanded state, as shown in FIG. 3. Responsively to the expanding of the prosthetic valve structure, valve leaflet frame 40, valve leaflets 44, upper skirt frame 36, and lower skirt frame 46 also expand from their respective collapsed states.

For some applications, annular valve ring 24 is coupled to a plurality of generally curved, prong-shaped anchors 32, for example, four to eight anchors, e.g., six anchors, as shown by way of illustration and not limitation in FIG. 1. In the expanded state of valve structure 20, as shown in FIG. 3, the anchors are disposed circumferentially and in concentric alignment with the annular valve ring. As shown in FIG. 3, the anchors project from the annular valve ring through the valve ring fabric sleeve 34. As the prosthetic valve structure transitions to a collapsed state, as shown in FIG. 2A, the anchors remain alongside respective segments of the annular valve ring to which each anchor is adjacently disposed in the expanded state of the prosthetic valve structure.

During implantation of prosthetic valve structure 20, a lower portion of the prosthetic valve structure is first advanced toward the ventricular surface of the native valve. Once the distal end of the catheter is positioned in the ventricle of the patient, the physician pushes distally on the upper valve pivot joints 30 in order to (1) expose annular valve ring portion 24 and the lower skirt frame 46 and lower skirt fabric 48 from within the catheter, and (2) in conjunction, expand the annular valve ring. As the annular valve ring expands, lower skirt frame 46, valve leaflet frame 40, and valve leaflets 44 passively (i.e., automatically) expand. As the physician expands the annular valve ring, each of the anchors remain disposed circumferentially with respect to the segment of the annular valve ring to which the anchor is adjacently disposed (as shown in FIG. 3). In such a manner, a space is created between each anchor and the respective segments of the annular valve ring to which each anchor is adjacently disposed.

By pulling proximally on the catheter and the tool coupled to prosthetic valve structure 20 disposed therein, the annular valve ring is positioned adjacent to a ventricular surface of the native valve. Once the valve ring portion is positioned adjacent to the ventricular surface, the physician rotates annular valve ring 24 (e.g., by rotating 30 degrees a tool coupled thereto) about an axis that runs between the native valve from the atrium to the ventricle (which during implantation of the valve, is typically approximately aligned with longitudinal axis 10 of the valve). During this rotation, portions of native chordae tendineae 60 are grasped and placed between each anchor and the respective segment of the annular valve ring to which the anchor is adjacently disposed, as shown in FIG. 4. This grasping of the leaflets provides supplemental support to the prosthetic valve during and following implantation thereof. Alternatively or additionally, support is provided to the prosthetic valve by the upper and lower skirts, and/or by ring 24.

In conjunction with the grasping of the chordae tendineae, the prosthetic valve is secured in place. The physician then pulls the catheter proximally in order to expose upper skirt frame 36 and upper skirt fabric 38 from within the catheter. The skirt then expands over the atrial surface of the native valve in order to create a flush coupling between the prosthetic valve and the native valve.

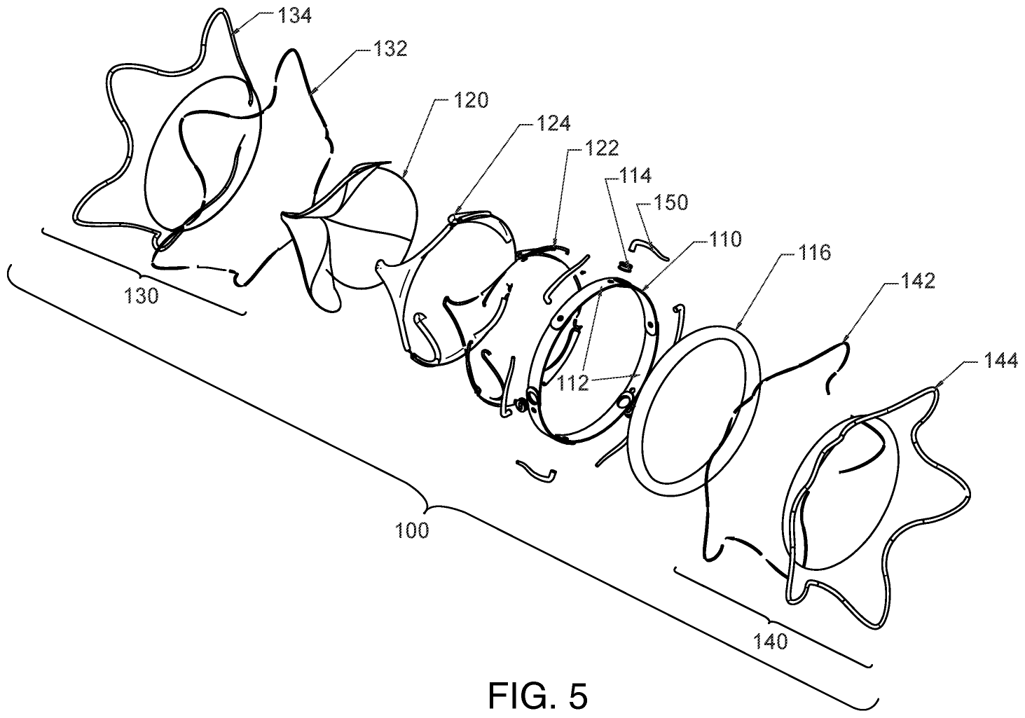

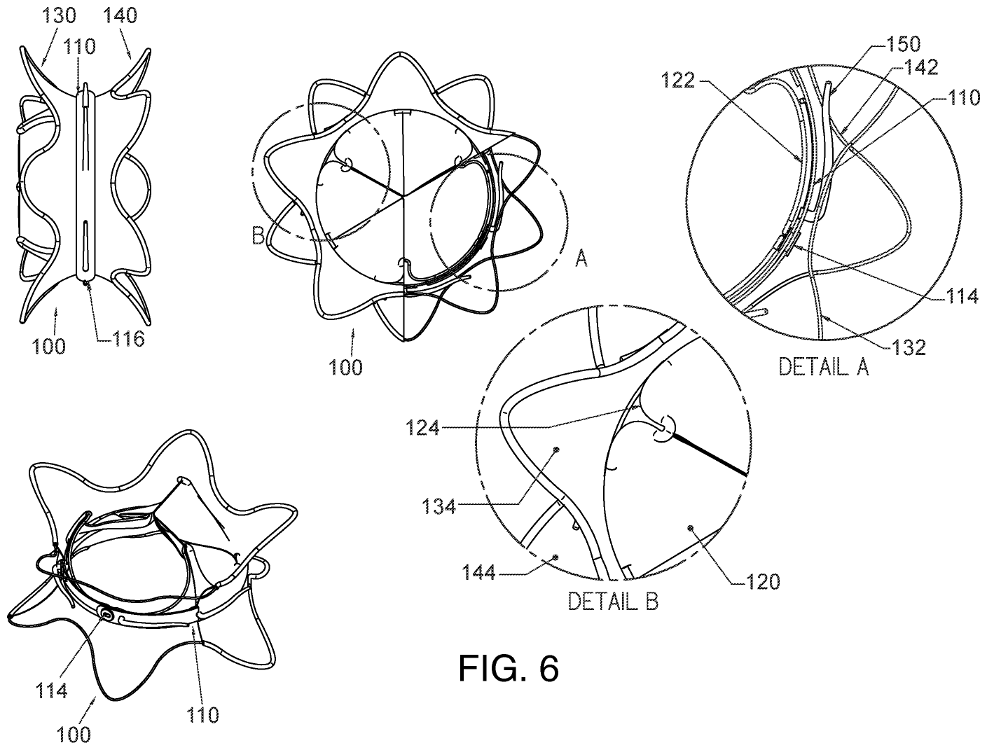

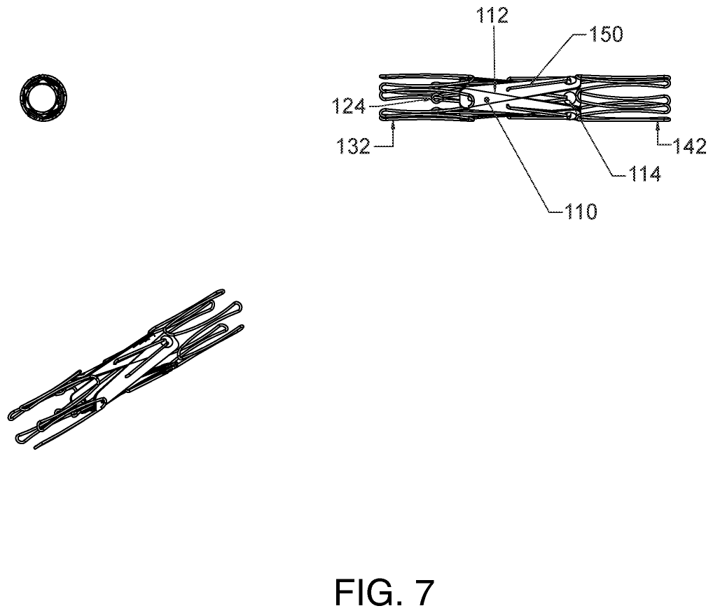

The following description, mutatis mutandis, originates from U.S. Provisional Patent Application 61/283,819 to HaCohen, which was incorporated by reference into the original specification of U.S. Ser. No. 12/961,721 to HaCohen:
Reference is now made to FIGS. 5-7 which are schematic illustrations of an expandable and collapsible prosthetic valve 100, in accordance with some applications of the present invention. The prosthetic valve is configured for implantation in and replacement of a native atrioventricular valve of a patient. Typically, the prosthetic valve is configured for implantation in and replacement of a native mitral valve of the patient. The prosthetic valve comprises an annular valve ring 110 (as shown in the exploded view of FIG. 5), which comprises a plurality of curved metal segments 112 and a plurality of pivot joints 114 which facilitate the collapsing and expanding of the prosthetic valve. The annular valve ring 110 is typically surrounded by a valve ring fabric sleeve 116 comprising a braided mesh of fabric, e.g., Dacron. This sleeve promotes fibrosis following implantation of the prosthetic valve in the native valve of the patient. The annular valve ring 110 is coupled to a plurality of valve leaflets 120. The valve leaflets are coupled to a flexible valve leaflet frame 122 (e.g., comprising nitinol by way of illustration and not limitation), which is, in turn, coupled to a valve leaflet frame fabric 124. Typically, the valve leaflet frame fabric (e.g., a fabric comprising Dacron) is coupled to (for example, sutured to) the valve ring fabric sleeve.
For embodiments in which the prosthetic valve is designated to replace the native mitral valve of the patient, the prosthetic valve comprises three artificial or tissue-based leaflets 120, as shown, which are coupled to the inner perimeter of the annular valve ring. It is to be noted that although these leaflets are shown by way of illustration and not limitation, as being a product of Neovasc Medical Ltd., the leaflets may comprise any other leaflets known in the art.
The annular valve ring portion 110 of the prosthetic valve is coupled: (1) at a first surface thereof to an upper skirt 140, which comprises an upper skirt fabric 144 coupled to a flexible upper skirt frame 142, and (2) at a second surface thereof to a lower skirt 130, which comprises a lower skirt fabric 134 coupled to a flexible lower skirt frame 132. Typically, the upper and lower frames comprise a flexible material, e.g., nitinol by way of illustration and not limitation. Typically, when the prosthetic valve is implanted in the expanded state, as shown in FIG. 6:
(a) the annular valve ring portion is configured to be disposed at a ventricular surface of the native valve,
(b) the upper skirt is designated to rest against an atrial portion of the native mitral valve, and
(c) the lower skirt is designated to rest against a ventricular surface of the native valve and to push radially the native leaflets of the native valve.
FIG. 5 shows the components of the valve in an exploded view. Each segment 112 of the annular valve ring is coupled at its respective ends to respective ends of adjacent segments via a hinge, or a valve pivot joint 114. The pivot joints of the ring portion enable the entire prosthetic valve to pivot into a collapsed state, as shown in FIG. 7. Typically, the valve comprises: (a) three “upper” valve pivot joints which are disposed at 120 degrees along the annular valve ring and are near the upper skirt frame, as shown in FIG. 7; and (b) three “lower” valve pivot joints also disposed with a separation therebetween of 120 degrees, alternating with the upper valve pivot joints. The lower valve pivot joints are near the lower skirt frame, as shown in FIG. 7). The upper valve pivot joints are exposed at a proximal portion of the valve in a collapsed state (i.e., adjacent to an upper skirt region of the prosthetic valve, as shown in FIG. 7). The pivot joints enable the prosthetic valve to assume a diameter of 6 mm (by way of illustration and not limitation) in its collapsed state, as shown in FIG. 7. In the collapsed state, the valve leaflet frame, the valve leaflets, the upper skirt, and the lower skirt are also collapsed.
In order to deploy the prosthetic valve inside the heart, the physician distally pushes on the upper pivot joints using a pushing tool. The pushing of the upper pivot joints enables the annular valve ring to expand radially in order for the prosthetic valve to assume an expanded state, as shown in FIG. 6. Responsively to the expanding of the prosthetic valve, the valve leaflet frame, the valve leaflets, the upper skirt, and the lower skirt also expand from their respective collapsed states.
The annular valve ring is coupled to a plurality of generally curved, prong-shaped anchors 150, e.g., 6 as shown by way of illustration and not limitation in FIG. 5. In an expanded state, as shown in FIG. 6, the anchors are disposed circumferentially and in concentric alignment with the annular valve ring. As shown in FIG. 6, the anchors project from the annular valve ring through the valve ring fabric sleeve. As the prosthetic valve transitions to a collapsed state, as shown in FIG. 7, the anchors remain alongside respective segments of the annular valve ring to which each anchor is adjacently disposed in the expanded state of the prosthetic valve.
During implantation of the prosthetic valve, a lower portion of the prosthetic valve is first advanced toward the ventricular surface of the native valve. Once the distal end of the catheter is positioned in the ventricle of the patient, the physician pushes distally on the upper valve pivot joints in order to (1) expose the annular valve ring portion and the lower skirt from within the catheter, and (2) in conjunction, expand the annular valve ring. As the annular valve ring expands, the lower skirt, the valve leaflet frame, and the valve leaflets passively expand. As the physician expands the annular valve ring, each of the anchors remain disposed circumferentially with respect to the segment of the annular valve ring to which the anchor is adjacently disposed (as shown in FIG. 6). In such a manner a space is created between each anchor and the respective segments of the annular valve ring to which each anchor is adjacently disposed.
By pulling proximally on the catheter and the tool coupled to the prosthetic valve disposed therein, the annular valve ring is positioned adjacent to a ventricular surface of the native valve. Once the valve ring portion is positioned adjacent to the ventricular surface, the physician rotates the annular valve ring (e.g., by rotating 30 degrees a tool coupled thereto) about an axis that runs between the native valve from the atrium to the ventricle. During this rotation, portions of the native chordeae tendineae are grasped and placed between each anchor and the respective segment of the annular valve ring to which the anchor is adjacently disposed. This grasping of the leaflets provides supplemental support to the prosthetic valve during and following implantation thereof.
In conjunction with the grasping of the chordeae tendineae, the prosthetic valve is secured in place. The physician then pulls the catheter proximally in order to expose the upper skirt from within the catheter. The skirt then expands over the atrial surface of the native valve in order to create a flush coupling between the prosthetic valve and the native valve.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a prosthetic valve that is designated for implantation at a native heart valve of a patient, comprising:
a valve ring having a plurality of ring segments, each of the segments being hingedly coupled to at least one adjacent segment at a pivot joint,
the valve ring being configured:
to be placed adjacent to a surface of the native heart valve, the prosthetic valve having been coupled to the valve ring,
in an expanded state thereof, to define a ring, all of the pivot joints being disposed in a plane that is perpendicular to a longitudinal axis of the ring, and
to be foldable from the expanded state into a shape that has a generally circular cross-section that defines and surrounds at least in part a central lumen, by folding the segments with respect to each other, at the pivot joints.

2. The apparatus according to claim 1, wherein the segments of the ring are configured to become at least partially twisted due to the ring being folded from the expanded state.

3. The apparatus according to claim 1, wherein the prosthetic valve includes a trileaflet valve, and wherein the ring has a number of ring segments that is a multiple of six.

4. The apparatus according to claim 3, wherein the ring has exactly six segments.

5. The apparatus according to claim 1, wherein the prosthetic valve includes a bileaflet valve, and wherein the ring has a number of ring segments that is a multiple of four.

6. The apparatus according to claim 5, wherein the ring has exactly four segments.

7. A method for use with a prosthetic valve that is designated for implantation at a native heart valve of a patient, comprising:
placing in a vicinity a surface of the native heart valve, a valve ring that is coupled to the valve, while the valve ring is in a folded state thereof,
the ring having a plurality of ring segments, each of the segments being hingedly coupled to at least one adjacent segment at a pivot joint,
in the folded state thereof, the ring having a shape that has a generally circular cross-section that defines and surrounds at least in part a central lumen;
expanding the ring such that all of the pivot joints become disposed in a plane that is perpendicular to a longitudinal axis of the ring, by applying a force to at least some of the pivot joints; and
when the ring is in an expanded state thereof, positioning the ring adjacent to a surface of the native valve.

8. The method according to claim 7, wherein applying the force to some of the pivot joints comprises pushing on pivot joints that are disposed on a proximal side of the ring, while the ring is in the folded state thereof.

9. The method according to claim 7, wherein the native valve includes a native mitral valve, and wherein positioning the ring adjacent to the surface of the valve comprises positioning the ring adjacent to a ventricular surface of the native mitral valve.

10. Apparatus for use with a prosthetic valve that is designated for implantation at a native mitral valve of a patient, comprising:
an annular ring configured to be placed at a ventricular surface of the native mitral valve, the prosthetic valve having been coupled to the annular ring; and
at least one anchor disposed circumferentially with respect to the annular ring so as to define a space between the anchor and the annular ring.

11. The apparatus according to claim 10, wherein the anchor is configured to grasp a portion of native chordae tendineae of a heart of the patient by the annular ring being rotated.

12. The apparatus according to claim 10, wherein the annular ring is configured to be collapsible.

13. The apparatus according to claim 12, wherein the ring comprises a plurality of ring segments, each of the segments being hingedly coupled to at least one adjacent segment at a pivot joint, and wherein the ring is configured to be collapsed by folding the segments with respect to each other, at the pivot joints.

14. A method, comprising:
positioning an annular ring portion of a prosthetic valve structure at a ventricular surface of a native heart valve of a patient; and
grasping a portion of native chordae tendineae of a heart of the patient by rotating the annular ring portion of the prosthetic valve structure.

15. The method according to claim 14, wherein grasping the portion of the native chordae tendineae comprises facilitating placing the portion of the native chordae tendineae in a space between a segment of the annular ring portion and an anchor disposed circumferentially with respect to the segment of the annular ring portion.

16. The method according to claim 14, wherein positioning the annular ring portion comprises:
transcatheterally advancing the prosthetic valve structure toward the native valve of the patient in a collapsed state thereof; and
expanding the prosthetic valve structure from the collapsed state.

17. The method according to claim 16, wherein expanding the prosthetic valve structure comprises pivoting a plurality of segments of the annular ring portion at respective pivot joints that couple together adjacent segments of the annular ring portion by distally pushing one or more of the pivot joints in a manner in which the ring portion expands radially.

18. A prosthetic heart valve for implantation at a native heart valve of a heart, the prosthetic heart valve configured to assume a collapsed state for transcatheter delivery and an expanded state, the prosthetic heart valve comprising:
a valve ring, which comprises metal segments, wherein (i) when the prosthetic heart valve is in its collapsed state, the valve ring is in a collapsed state, and (ii) when the prosthetic heart valve is in its expanded state, the valve ring is in an expanded state such that the valve ring is cylindrical;
a valve ring fabric sleeve, which is coupled to the valve ring;
a leaflet frame, which is disposed within the valve ring;
a plurality of prosthetic leaflets, which are coupled to the leaflet frame, and configured to facilitate upstream-to-downstream bloodflow longitudinally through the prosthetic heart valve; and
an upper skirt, which:
(i) comprises (a) an upper skirt fabric, which is coupled to the valve ring fabric sleeve, and (b) a flexible upper skirt frame, which is coupled to the upper skirt fabric such that the upper skirt fabric separates a downstream end of the upper skirt frame from an upstream end of the metal segments of the valve ring, and
(ii) when the prosthetic heart valve is in its expanded state, extends radially outward from the valve ring in an upstream direction and is configured to rest against an atrial surface of the heart.

19. The prosthetic heart valve according to claim 18, further comprising a valve leaflet frame fabric, which is coupled to the leaflet frame.

20. The prosthetic heart valve according to claim 19, wherein the valve leaflet frame fabric is coupled to the valve ring fabric sleeve.

21. The prosthetic heart valve according to claim 18, wherein the valve ring is configured such that the metal segments are curved when the valve ring is in its expanded state.

22. The prosthetic heart valve according to claim 18, wherein the valve ring fabric sleeve surrounds the valve ring.

23. The prosthetic heart valve according to claim 18, wherein the flexible upper skirt frame comprises Nitinol.

24. The prosthetic heart valve according to claim 18, wherein, when the prosthetic heart valve is in its expanded state, the upper skirt frame is shaped so as to define upstream peaks and downstream troughs, and wherein the downstream troughs define the downstream end of the upper skirt frame.

25. The prosthetic heart valve according to claim 18, further comprising a lower skirt, which comprises a lower skirt fabric coupled to a flexible lower skirt frame.

26. The prosthetic heart valve according to claim 18, wherein the native heart valve is a native mitral valve, and wherein the upper skirt is configured to rest against the atrial surface within a left atrium of the heart when the prosthetic heart valve is in its expanded state.

27. The prosthetic heart valve according to claim 18, further comprising a plurality of anchors coupled to the metal segments of the valve ring.

28. A method comprising:
transcatheterally advancing a prosthetic heart valve toward a native heart valve of a heart while the prosthetic heart valve is in a collapsed state and a valve ring of the prosthetic heart valve is in a collapsed state; and
at the native heart valve, expanding the prosthetic heart valve to an expanded state in which:
the valve ring is in an expanded state such that the valve ring is cylindrical, wherein a valve ring fabric sleeve is coupled to the valve ring,
a leaflet frame is disposed within the valve ring, and a plurality of prosthetic leaflets, which are coupled to the leaflet frame, facilitate upstream-to-downstream bloodflow longitudinally through the prosthetic heart valve, and
an upper skirt extends radially outward from the valve ring in an upstream direction and rests against an atrial surface of the heart, wherein the upper skirt includes (a) an upper skirt fabric, which is coupled to the valve ring fabric sleeve, and (b) a flexible upper skirt frame, which is coupled to the upper skirt fabric such that the upper skirt fabric separates a downstream end of the upper skirt frame from an upstream end of metal segments of the valve ring.

29. The method according to claim 28, wherein the prosthetic heart valve further includes a valve leaflet frame fabric, which is coupled to the leaflet frame.

30. The method according to claim 29, wherein the valve leaflet frame fabric is coupled to the valve ring fabric sleeve.

31. The method according to claim 28, wherein expanding the prosthetic heart valve comprises expanding the prosthetic heart valve such that the metal segments of the valve ring are curved.

32. The method according to claim 28, wherein the valve ring fabric sleeve surrounds the valve ring.

33. The method according to claim 28, wherein the flexible upper skirt frame includes Nitinol.

34. The method according to claim 28, wherein expanding the prosthetic heart valve comprises expanding the prosthetic heart valve such that the upper skirt frame is shaped so as to define upstream peaks and downstream troughs, and the downstream troughs define the downstream end of the upper skirt frame.

35. The method according to claim 28, the prosthetic heart valve further includes a lower skirt, which includes a lower skirt fabric coupled to a flexible lower skirt frame.

36. The method according to claim 28, wherein the native heart valve is a native mitral valve, and wherein expanding the prosthetic heart valve at the native heart valve comprises expanding the prosthetic heart valve at the native mitral valve.

37. The method according to claim 28, further comprising anchoring the prosthetic heart valve to the heart using a plurality of anchors coupled to the metal segments of the valve ring.
